(19) United States
(12) Patent Application Publication
(10) Pub. No.: US 2012/0330133 A1
(43) Pub. Date: Dec. 27, 2012

(54) NONSURGICAL DETERMINATION OF ORGAN TRANSPLANT CONDITION

(76) Inventor: Edward R. Flynn, Albuquerque, NM (US)

(21) Appl. No.: 13/581,789

(22) PCT Filed: Mar. 16, 2011
(86) PCT No.: PCT/US11/28746

§ 371 Date: Aug. 29, 2012

Related U.S. Application Data
(60) Provisional application No. 61/314,370, filed on Mar. 16, 2010.

(51) Int. Cl. A61B 5/055; G01R 35/00

(52) U.S. Cl. 600/420; 324/202

(57) ABSTRACT

A Superconducting Quantum Interference Device (SQUID) magnetic sensor system and method can image organic transplant condition, such as status, acceptance, or rejection, in-vivo. This represents a major advane in transplant imaging technology with a new market for biomagnetic sensor devices. In-vivo transplant condition determination provides a greater range of imaging methodologies over existing methods in sensitivity, and enables early detection of rejection with the ability to determine the need for anti-rejection drugs.

### TECHNICAL FIELD

This invention relates to organ transplants and, in particular, to a nonsurgical method and system for the determination of organ transplant condition such as acceptance or rejection.

### BACKGROUND ART

There are about 52,000 people in the United States on waiting lists for kidney transplants. In addition, 60,000 people die each year of kidney disease. Between 1996 and 1998, 94,000 kidney transplants were done in the United States. The number of rejected kidneys in 1996 was 6% from live donors and 12% from dead donors. Other reports mention that one out of three people receiving kidney transplants have at least one kidney rejection episode. A Johns Hopkins study in 2002 mentions 12,000 kidneys are transplanted annually with 5,000 of these from live donors. However, one-third of these transplants find that the donors are not good matches.

The large percentage of kidney rejections is due to actions of the immune system. This problem is normally minimized by careful selection of donors to match the recipients, followed by the application of a form of chemotherapy to reduce the immune system response to the newly transplanted organ. The chemotherapy drugs normally used are cyclosporine and, more recently, daclizumab. These chemotherapy drugs are also accompanied by immune-suppressing steroids. Another method to minimize rejection is to filter out donor-specific antibodies from the blood of the patient; this is referred to as plasmapheresis.

These methods are often insufficient, resulting in rejection of the organ by the recipient. In acute rejection, which can begin within 24 hours of transplantation and occurs over days to weeks, the immune system recognizes certain proteins on the surface of cells in the transplanted organ as ligands and prepares antibodies to attack the cells of the organ. The immune system produces B cell lymphocytes that generate antibodies that attach to these ligands to destroy them, as well as T cells (T lymphocytes) which react against these foreign cell-surface proteins on the transplanted cells. The most important of the proteins recognizing as foreign cells are the major histocompatability complex (MHC) that appear on all invertebrate cells and are the human-leukocyte-associated antigens (HLA antigens). The presence of these lymphocytes, primarily mediated by T cells, indicates that the organ is being rejected. The T cells recognize the MHC proteins that have bound to the foreign proteins on the surface of the host cells and they also recognize foreign MHC proteins that may be present. The antibodies CD* and CD4 are co-receptors on T cells where CD8 is expressed primarily on cytotoxic T cells recognizing Class I MHC proteins and CD4 is expressed primarily on helper T cells and Class II MHC proteins.

There are a large number of lymphocyte cells in the body, ˜10¹², that primarily reside in the lymphatic system and the lymphoid organs (thymus, spleen and appendix). Lymphocytic cells are not normally present in any amounts in other organs, but on recognition of a foreign substance they exponentially multiply and invade the organ. The patient will suffer with fever or other responses to this occurring and a biopsy of the transplant is typically made to determine the presence of lymphocytes through microscopic observation or other means. There is an initial period of inflammation after the transplant due to the surgery damage itself which must also be taken into account in any studies of this type.

Organ transplant monitoring by biopsy is painful, risks infection, and causes morbidity. Therefore, a need remains for a system and method for the nonsurgical determination of organ transplant acceptance.

### DISCLOSURE OF INVENTION

This application is related to U.S. provisional application 61/314,370, filed Mar. 16, 2010, which application is incorporated herein by reference. The present invention provides a system and method for nonsurgical determination of organ transplant condition such as acceptance or rejection. The system comprises a magnetic field detector, such as a superconducting quantum interference device sensor, comprising a magnetic pulser, adapted to apply a uniform magnetizing pulse field to a transplanted organ of a patient placed on a measurement stage; and a remnant magnetic field detector, adapted to detect and image the residual magnetic field produced by the applied pulsed field. The magnetic pulser can comprise a pair of Helmholtz coils. The remnant magnetic field detector can comprise an array of gradiometers. An example method according to the present invention comprises providing a superconducting quantum interference device sensor system; injecting a plurality of antibody-labeled magnetic nanoparticles into a patient placed on a measurement stage for specific binding to the transplanted organ; applying a uniform magnetizing pulse field to magnetize the nanoparticles injected into the patient; and detecting the residual magnetic field of the magnetized nanoparticles thereby providing an image of the nanoparticles bound to the transplanted organ of the patient. For example, the transplanted organ can comprise a kidney. The antibody-labeled magnetic nanoparticle can comprise a magnetic core coated with a biocompatible coating to which is attached at least one specific antibody. For example, the magnetic core can comprise a ferromagnetic material, such as iron oxide. For example, the antibody-labeled magnetic nanoparticles can comprise antibodies that specifically bind to T cells.

### BRIEF DESCRIPTION OF DRAWINGS

The accompanying drawings, which are incorporated in and form part of the specification, illustrate the present invention and, together with the description, describe the invention. In the drawings, like elements are referred to by like numbers.

FIG. 1 is a photograph of a Superconducting Quantum Interference Device (SQUID) sensor system that can be used for nonsurgical determination of organ transplant acceptance.

FIG. 2 is a schematic illustration of a SQUID sensor system for nonsurgical determination of organ transplant acceptance in humans.

FIG. 3 is a schematic illustration of the magnetic nanoparticles used for calibration and nonsurgical determination of organ transplant acceptance.

FIG. 4 is a photograph of full sized kidney phantom containing two sources of nanoparticles.

FIG. 5A is a Transmission Electron Microscope (TEM) image of the nanoparticles used for SQUID sensor imaging. FIG. 5B is a T-cell with attached nanoparticles.

FIG. 6 is a graph of incubation curves for the CD3 antibody connecting to two T-cell lines.

FIG. 7 is a graph showing the sensitivity of the method at conditions in a kidney transplant undergoing rejection.

FIG. 8A is a bar chart showing the magnetic signal obtained from a fixed number of U937 cells as a function of dilution with real human blood. FIG. 8B shows microphotographs of Prussian blue stains of these same samples.

FIGS. 9A and 9B are H & E-stained histological sections of isogenic mouse skin grafts.

### MODES FOR CARRYING OUT THE INVENTION AND INDUSTRIAL APPLICABILITY

The present invention can use a Superconducting Quantum Interference Device (SQUID) magnetic sensor for the nonsurgical determination of organ transplant condition such as status, acceptance, or rejection. The SQUID sensor is a highly sensitive instrument that can detect magnetic fields created by clusters of magnetic nanoparticles. The SQUID sensor enables non-invasive determination of organ transplant acceptance. Additionally, the non-invasive nature of the technology allows more frequent monitoring of the patient, compared to biopsy. The physician can also use this technology to calibrate the level of medication if it appears that T cells have infiltrated the transplanted organ.

T cells congregate in specific areas of the organ. Biopsy only removes a small sample of tissue from the organ and does not sample the organ as a whole. The present invention can enable the physician to image the entire organ. This allows a physician to assess what degree of organ rejection, if any, is occurring in the patient. This reduces the need for invasive biopsy procedures and enables the monitoring of an organ transplant for the effects of chemotherapy. For example, the ability to assess and quantify the population of CD8 T cells in a specific organ transplant can complement and often replace the existing method of organ transplant monitoring (biopsy). The technology enables accurately assessing the immune system response to the organ transplant to determine if acute or chronic rejection is taking place. The invention can also provide the ability to monitor CD8 as well as CD4 T cells.

A biomagnetic SQUID sensor can be used together with antibody-labeled-magnetic nanoparticles to detect the buildup of clusters of excess lymphocytes in a transplanted organ. This system reduces the need for biopsies and provides a non-invasive method for monitoring the effectiveness of immune-suppressive drugs. This method easily identifies these lymphocyte cells. Reduction of biopsies is of great patient benefit since the biopsies are painful and there is reasonable chance for infection. Infection is of great concern since patients often have a reduced immune system response due to the chemotherapy. Thus, any method which can significantly eliminate the need for invasive procedures can have substantial impact on the patient's well being.

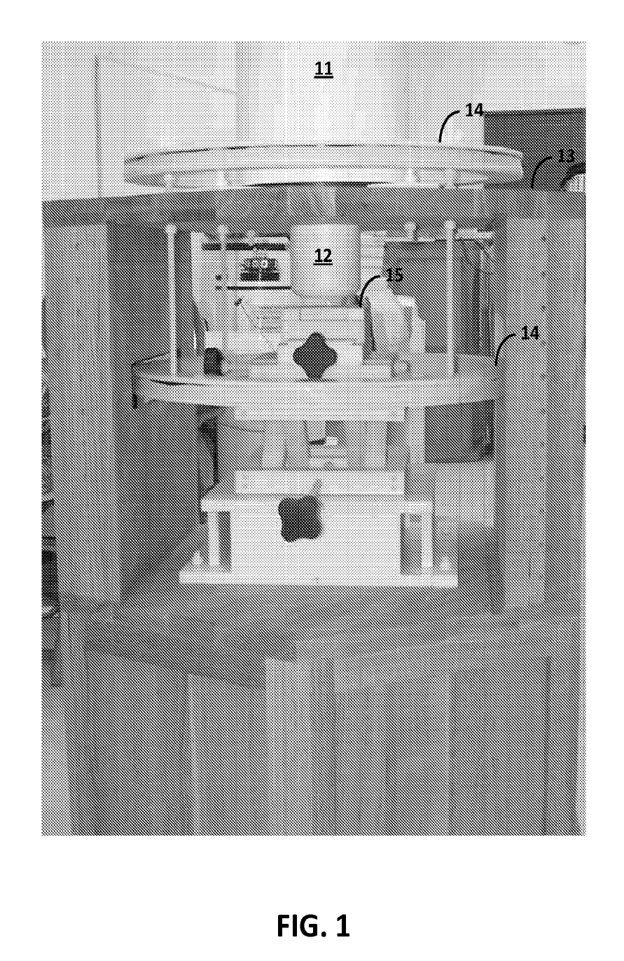

FIG. 1 shows an exemplary SQUID sensor with a liquid helium reservoir dewar 11 at the top of the picture. The sensor comprises a magnetic field pulser, adapted to apply a uniform magnetizing pulse field to a transplanted organ of a patient placed on a measurement stage, and a magnetic field detector, adapted to detect and image the residual magnetic field produced by the applied pulsed field. As an example, the magnetic field pulser can comprise two circular coils 14 forming a Helmholtz pair that can provide a magnetizing pulsed field for the nanoparticles. The uniform field produced by these coils can be varied but typically is 40 to 50 Gauss and the pulse length is typically 300-800 msec. As an example, the magnetic field detector can comprise SQUID 2^{nd}-order axial gradiometers that are contained in a snout 12 protruding through a support frame 13. There are seven gradiometers contained within this exemplary snout; one in the center and six in a circle of 2.15 cm radius. Each gradiometer is inductively coupled to a low temperature SQUID. In this example, a wooden frame supports the SQUID and the measurement platform as well as the magnetizing coils. The non-magnetic support system comprises a 3-dimensional stage 15 that can, for example, be constructed of plastic with no metal components. The upper two black knobs control the x-y stage movements over a +/−10 cm range and the lower knob is used to raise and lower the measurement stage over a 20 cm range. A sample holder can be inserted onto the stage that can contain nanoparticle samples, live cell samples, or live mice.

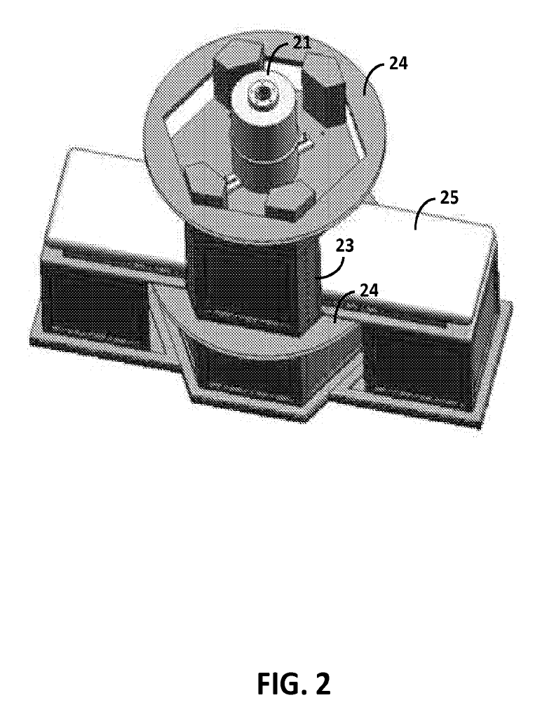

FIG. 2 shows an exemplary SQUID sensor that can be used for human organ transplant acceptance examinations. A wooden or other non-conductive structure 23 can be similar to the support frame shown in FIG. 1. The measurement stage can be replaced by a bed 25 for patient placement. Two larger Helmholtz coils 24 comprise the wooden circular forms above and below the bed. These larger coils can again be used to generate a uniform pulse field and magnetize magnetic nanoparticles that have been injected into the patient. The currents can be increased from those used in the system shown in FIG. 1 to again produce fields in the range of 40 to 50 Gauss. Similar to the system shown in FIG. 1, a SQUID dewar 21 with an array of magnetic gradiometers can be used to measure the residual magnetic field change produced by the magnetized nanoparticles.

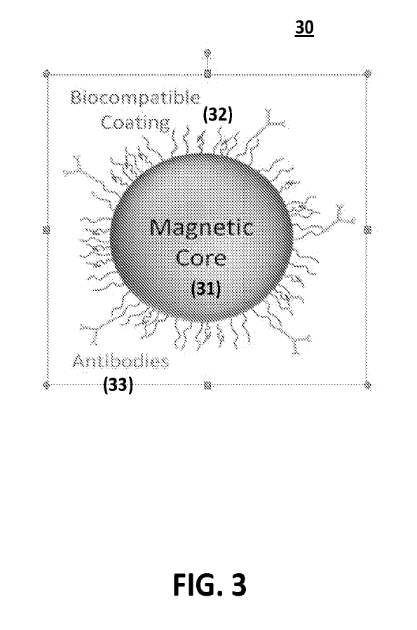

FIG. 3 is a schematic illustration of a magnetic nanoparticle 30 that can be used for calibration and in-vivo studies of human organ transplant acceptance. The center of the magnetic nanoparticle 30 can comprise of a magnetic core 31. For example, the core 31 can be iron-oxide of about 20-30 nanometers in diameter. This core 31 can be coated with a biocompatible coating 32, such as Dextran, carboxyl, or amine, to which is attached specific antibodies 33 to the transplanted organ. For example, the specific antibody can bind to a T-cell that is responsive to the organ transplant acceptance. The antibody can be specific to T-cell receptors on the surface of the T cell. One such specific antibody is a CD antibody, however other antibodies specific to organ transplant acceptance can be attached to the biocompatible surface through conjugation methods.

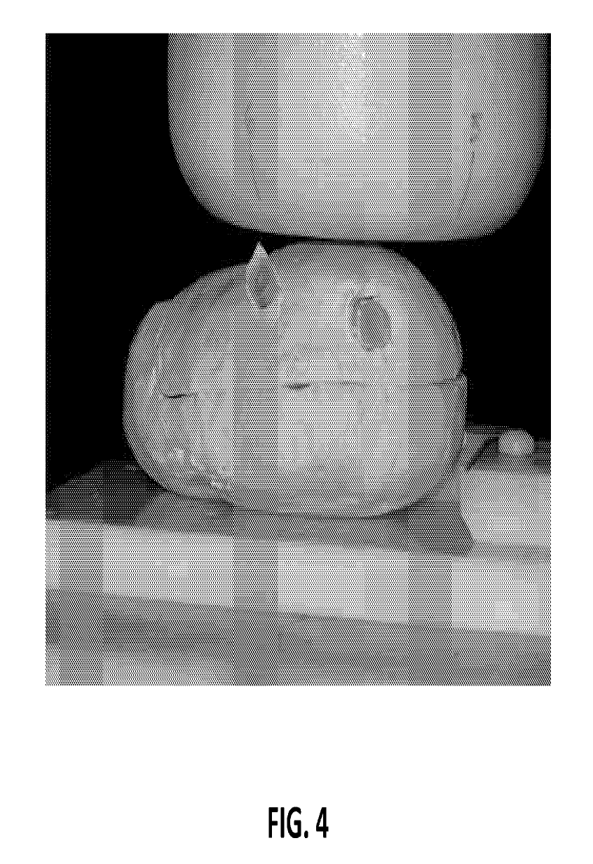

FIG. 4 is a photograph of full sized kidney phantom containing two sources of nanoparticles. Each source has 5.26×10¹⁰ Simag-1411 carboxyl coated nanoparticles conjugated to the antibody CD3 and attached to live T-Cells (Jurkat cell line). There are 8.22×10⁶ cells, each has 3×10⁴ nanoparticles (24 nm diameter) attached covering 21% of the available antigen sites.

Table 1 shows the comparison between physically measured locations of the live T-cells shown in the phantom of FIG. 4 with the spatial locations derived from the SQUID sensor array obtained from the magnetization of the magnetic nanoparticles on the cells.

TABLE 1

| | X (cm) | Y (cm) | Z (cm) | Moment (J-pT) |
|---|---|---|---|---|
| Source 1 | | | | |
| Measured | 1.4 ± 0.3 | −1.1 ± 0.3 | 5.5 ± 0.3 | 1.52 × 10⁵ |
| Imaged | 1.3 ± 0.1 | −1.0 ± 0.1 | 5.0 ± 0.05 | 1.45 × 10⁵ |
| Source 2 | | | | |
| Measured | −2.7 ± 0.3 | −1.5 ± 0.3 | 5.5 ± 0.3 | 1.60 × 10⁵ |
| Imaged | −2.7 ± 0.1 | −2.2 ± 0.1 | 5.0 ± 0.05 | 1.60 × 10⁵ |

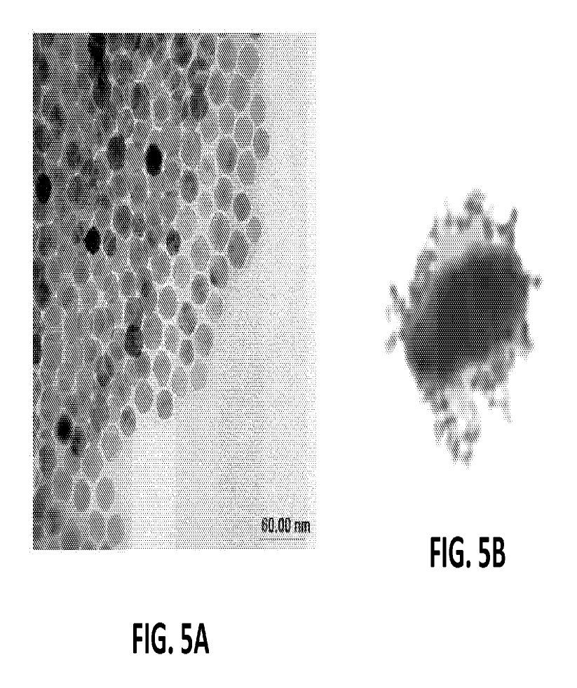

FIG. 5A is a Transmission Electron Microscope (TEM) image of the nanoparticles used for SQUID sensor imaging. FIG. 5B is a T-cell with attached nanoparticles. The nanoparticles are fairly uniform and roughly spherical with diameter of 25 nm; the cell diameter is approximately 10 microns in diameter with about ˜100,000 nanoparticles attached to it through CD2 antibodies.

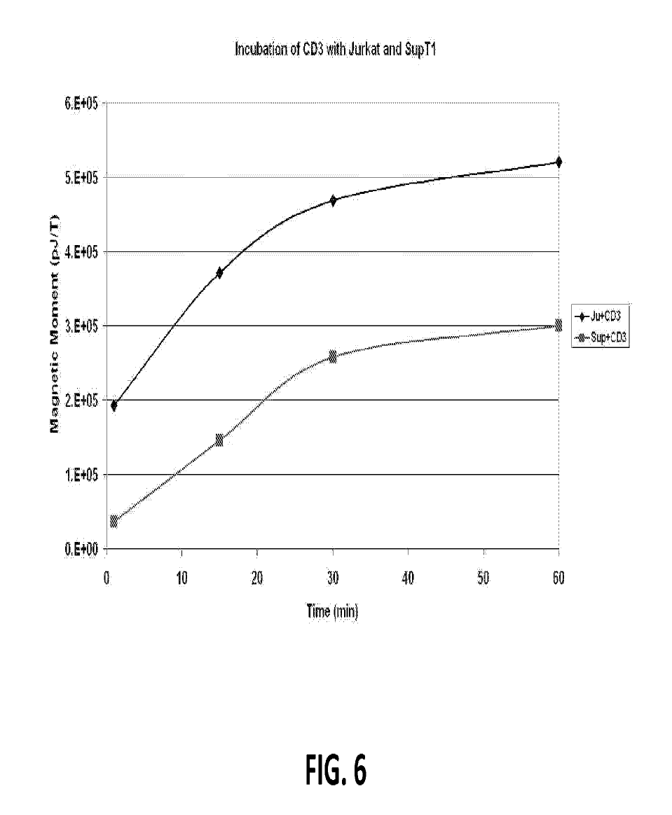

FIG. 6 shows incubation curves for the CD3 antibody connecting to two T-cell lines. The cell lines used were for two leukemia T-cells so that could be grown and their capabilities for attaching labeled magnetic nanoparticles measured. Non-leukemic cell lines should have similar characteristics as these. The curves show that the magnetic moments differ somewhat for different cell lines as expected for these two particular leukemia cells, with the Jurkat cells having a larger receptor number than the SupT1 cell line. These results indicate that the cells take up the particles in less than an hour.

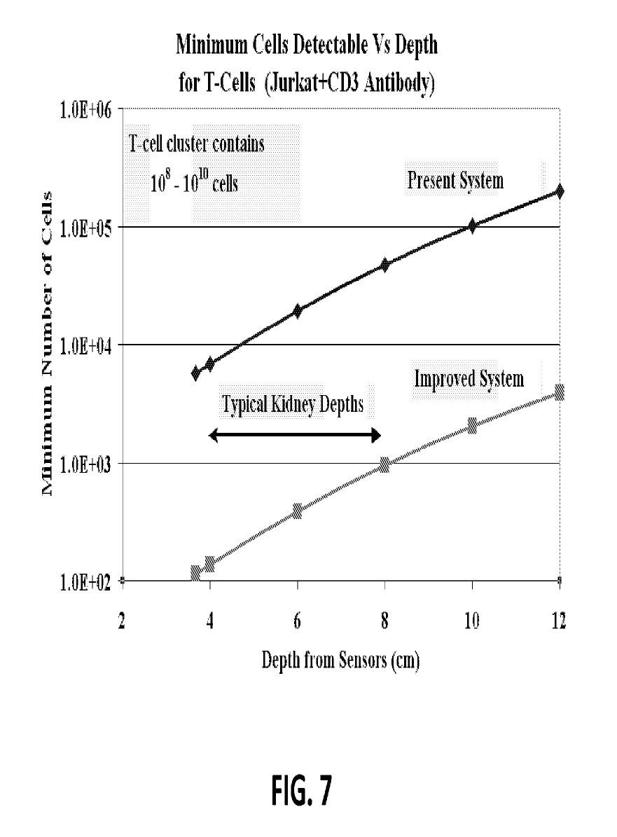

FIG. 7 shows an extrapolation the results of the T-cell experiments (using the Jurkat cell results) to conditions in a kidney transplant undergoing rejection. The kidney was assumed to be similar in shape to the phantom shown in FIG. 4 and to contain clusters of T-cells as in the vials inserted into the phantom, representing actual clusters of T-cells attacking the kidney. The upper curve represents the sensitivity for detecting T-cells as a function of distance from the sensors for the SQUID sensor system tested. The lower curve represents a SQUID system operating at optimal conditions with respect to sensor and background electromagnetic noise. The results indicate that at an average depth of T-cells in the kidney of approximately 6 cm, the tested system can detect about twenty thousand cells, whereas it is expected that a typical T-cell cluster in a rejected kidney may contain one hundred million or more cells.

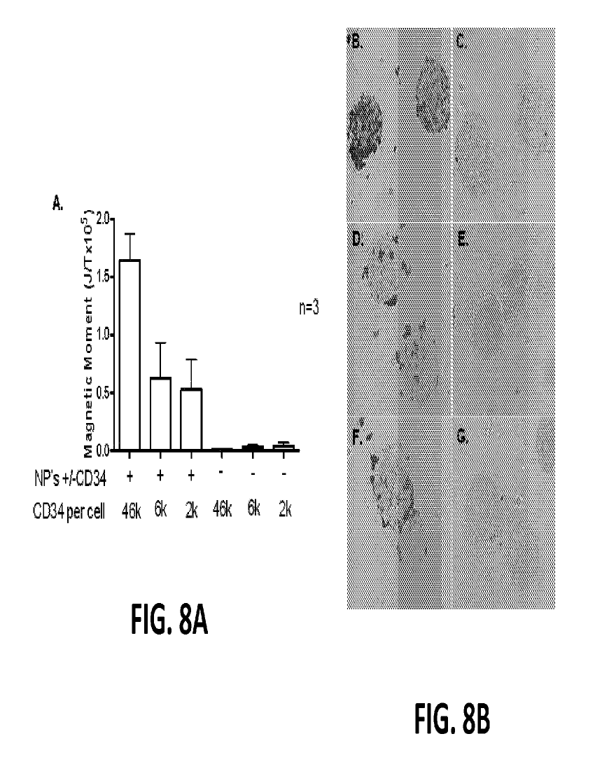

FIG. 8 shows the results of an investigation of the specificity of this targeting method by measuring the magnetic signal as a function of dilution of the cells. The bar chart in FIG. 8A shows the magnetic signal obtained from a fixed number of U937 cells (another T-cell leukemia line) as a function of dilution with real human blood. The microphotographs in FIG. 8B are Prussian blue stains of these same samples showing the reduction of the number of nanoparticles per cell as the dilution is increased. A variety of titration and other experiments have also been performed to determine the maximum site binding of SiMag (Chemicell, Berlin) and Ocean (Ocean Nanotech, Little Rock Arkansas) magnetic nanoparticles as well as to determine the saturation levels as a function of numbers of cells present.

A demonstration of the method of determining transplant rejection was carried out using an animal model in which skin transplants were made to mice of the same genetic background as the donor (a white mouse) and to different backgrounds (a black mouse). When these mice were injected with magnetic nanoparticles with antibodies directed for the T-cells that attack organs of unrelated donors, the white mice showed no sign of T-cells in the vicinity of the transplant whereas the black mice showed millions of the T-cells present; i.e., a sign of rejection of the transplant. This was verified by subsequent falling off of the transplant on the black mouse while the transplant on the white mouse integrated into the skin.

An animal model involving skin transplantation was used. In this model, normal mice have a patch of skin removed from the dorsal scapular region, then back or tail skin from a mouse of a different strain was applied to the exposed area (allogenic graft). Alternatively, the mouse had a section of skin from a genetically identical mouse applied as a control (isogenic graft). This transplant model was relatively simple to perform, and offered the advantage of allowing direct examination of graft success/rejection. Following these procedures, a skin patch from another animal was taken and applied in the same way and followed the same methods as developed for wound healing. After a fixed time, the mouse was injected with the nanoparticles conjugated with antibodies as developed in specific aim 3 for T-cells. The mouse was then placed under the SQUID system and magnetic remanence fields were measured. The mice were imaged at several time points during graft rejection, and following each SQUID imaging session, a small section of skin at the donor/recipient junction can be removed using a punch biopsy to confirm T cell infiltration.

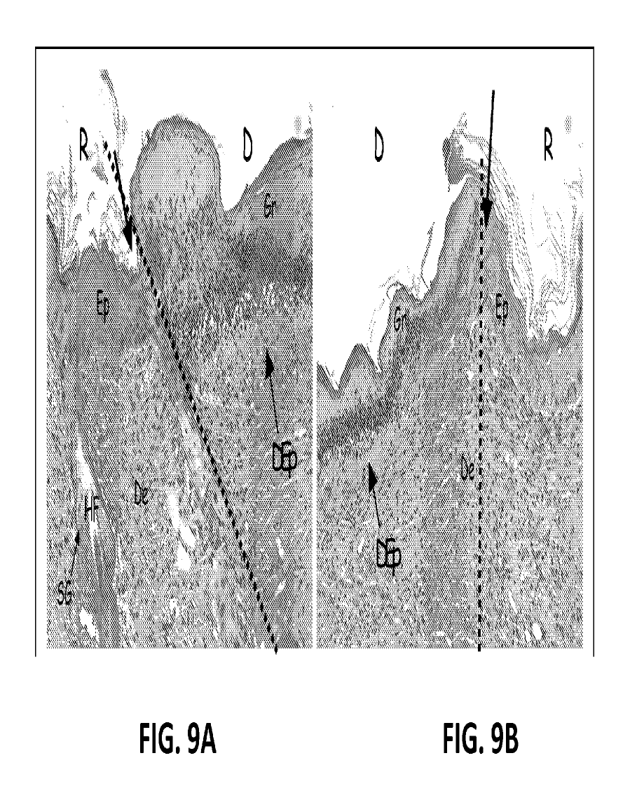

FIGS. 9A and 9B show H & E-stained histological sections of isogenic mouse skin grafts (where: Ep, recipient's endogenous epidermis; De, dermis; HF, hair follicle; SG, sebaceous gland). In these examples, donor back skin was grafted onto the back of a genetically identical recipient. After two weeks, the skin was harvested, and examined microscopically. The junction between donor and recipient skin (arrows) is shown in both panels (dotted lines separate recipient (R) skin from donor (D) skin). The donor skin appears to be re-epithelializing in both panels (DEp), underneath the graft (Gr).

The present invention has been described as a system and method for the nonsurgical determination of organ transplant acceptance. It will be understood that the above description is merely illustrative of the applications of the principles of the present invention, the scope of which is to be determined by the claims viewed in light of the specification. Other variants and modifications of the invention will be apparent to those of skill in the art.

What is claimed is:

1) A superconducting quantum interference device sensor system, comprising:
a) a magnetic pulser, adapted to apply a uniform magnetizing pulse field to a transplanted organ of a patient placed on a measurement stage; and
b) a remnant magnetic field detector, adapted to detect and image the residual magnetic field produced by a plurality of antibody-labeled magnetic nanoparticles injected into the patient for specific binding to T cells.

2) The system of claim 1, wherein the remnant magnetic field detector provides an image of the nanoparticles bound to the T cells on the transplanted organ of the patient.

3) A method for nonsurgical determination of organ transplant condition, comprising:
a) providing a superconducting quantum interference device sensor system comprising:
i) a magnetic pulser, adapted to apply a uniform magnetizing pulse field to a transplanted organ of a patient, and
ii) a remnant magnetic field detector, adapted to detect, measure, image, or a combination thereof, the residual magnetic field produced by the applied pulsed field;
b) injecting a plurality of magnetic nanoparticles, each labeled with a targeting agent such as an antibody or peptide, into the patient for specific binding to the transplanted organ;
c) applying the uniform magnetizing pulse field to magnetize the nanoparticles injected into the patient; and
d) detecting the residual magnetic field of the magnetized nanoparticles thereby providing an image of the nanoparticles bound to the transplanted organ of the patient.

4) The method of claim 3, wherein the magnetic pulser comprises a pair of Helmholtz coils.

5) The method of claim 3, wherein the remnant magnetic field detector comprises an array of gradiometers.

6) The method of claim 3, wherein the remnant magnetic field detector comprises an imaging means that solves an electromagnetic inverse problem.

7) The method of claim 3, wherein the transplanted organ comprises a kidney.

8) The method of claim 3, wherein the magnetic nanoparticles are labeled with an antibody that specifically binds to T-cells present in or near the transplanted organ.

9) The method of claim 3, wherein the magnetic nanoparticle comprises a magnetic core coated with a biocompatible coating to which is attached at least one specific antibody.

10) The method of claim 9, wherein the magnetic core comprises a ferromagnetic material.

11) The method of claim 10, wherein the ferromagnetic material comprises iron oxide.

12) The method of claim 9, wherein the magnetic core is less than 30 nanometers in diameter.

13) The method of claim 9, wherein the biocompatible coating comprises Dextran, carboxyl, or amine.

14) The method of claim 9, wherein the at least one specific antibody comprises a T cell specific antibody.

15) The method of claim 14, wherein the T cell specific antibody comprises a CD antibody.

16) A method to calibrate a superconducting quantum interference device sensor system, comprising:
a) providing a superconducting quantum interference device sensor system comprising:
i) a magnetic pulser, adapted to apply a uniform magnetizing pulse field to a phantom, comprising a known amount of antibody-labeled magnetic nanoparticles, placed on a measurement stage, wherein the antibody-labeled magnetic nanoparticles are bound to a specific T cell line, and
ii) a remnant magnetic field detector, adapted to detect and image a residual magnetic field produced by the applied pulsed field;
b) applying the uniform magnetizing pulse field to magnetize the nanoparticles in the phantom placed on the measurement stage; and
c) detecting the residual magnetic field of the magnetized nanoparticles thereby providing a sensitivity calibration for a organic transplant model.

17) A method as in claim 16, wherein the antibody-labeled magnetic nanoparticle comprises a magnetic core that comprises a ferromagnetic material.

18) A method as in claim 17, wherein the ferromagnetic material comprises iron oxide.

19) A method as in claim 16, wherein the antibody-labeled magnetic nanoparticle comprises a magnetic core that is less than 30 nanometers in diameter.

20) A method as in claim 16, wherein antibody-labeled magnetic nanoparticle comprises a biocompatible coating comprising Dextran, carboxyl, or amine.

21) A method as in claim 16, wherein the T cell specific antibody comprises a CD antibody.
